(19) United States
(12) Patent Application Publication
(10) Pub. No.: US 2022/0208317 A1
(43) Pub. Date: Jun. 30, 2022

(54) IMAGE CONTENT EXTRACTION METHOD AND IMAGE CONTENT EXTRACTION DEVICE

(71) Applicant: Industrial Technology Research Institute, Hsinchu (TW)

(72) Inventors: Cheng-Yi Yang, Hsinchu County (TW); Cheng-Liang Lin, Hsinchu County (TW); Hsing-Chen Lin, Taichung City (TW)

(73) Assignee: Industrial Technology Research Institute, Hsinchu (TW)

(21) Appl. No.: 17/136,036

(22) Filed: Dec. 29, 2020

(51) Int. Cl. G16H 10/60; G16H 30/20; G06F 16/583

(52) U.S. Cl. CPC G16H 10/60; G06F 16/5838; G06F 16/5846; G16H 30/20

(57) ABSTRACT

An image content extraction method and an image content extraction device are disclosed. The method includes the following. An image file is obtained; the image file is analyzed to obtain distribution information of at least one grid in an image frame corresponding to the image file; template information is determined according to the distribution information of the at least one grid; text information is extracted from the image file according to the template information; and integration information related to a medical record of a user is generated according to the text information.

### TECHNICAL FIELD

The disclosure relates to an image analysis technology, and in particular to an image content extraction method and an image content extraction device.

### BACKGROUND

Generally, after a patient receives medical treatment due to a specific physiological condition, the doctor or the hospital might provide a certificate of diagnosis. Later, the certificate of diagnosis may be used for applying for an insurance claim, taking a leave, receiving inter-hospital medical treatment, or transferring to another hospital, etc., serving as a proof of the patient's medical record and past medical history of physical or mental illnesses. However, under the current medical system, most of the certificates of diagnosis issued by hospitals are printed, and the form formats of the certificates of diagnosis of different hospitals are different. Therefore, when the certificate of diagnosis is required later (such as when the patient applies for an insurance claim), a party (such as an insurance company) is often required to receive the printed certificates of diagnosis and conduct complicated operations including a manual review. The process results in a waste of human resources for the reviewing party, and/or a compromised experience in using a printed certificate of diagnosis for the patient.

However, since printed certificates of diagnosis with a hospital's stamp is still necessary in use (for example, for anti-counterfeiting); therefore, at this stage, those skilled in the art in the field are committed to developing a method for effectively improving the efficiency of the use of printed certificates of diagnosis.

### SUMMARY

The disclosure provides an image content extraction method and an image content extraction device, which may effectively improve the efficiency of the use of printed certificates of diagnosis.

The embodiment of the disclosure provides an image content extraction method, which includes the following. An image file is obtained; the image file is analyzed to obtain distribution information of at least one grid in an image frame corresponding to the image file; template information is determined according to the distribution information of the at least one grid; text information is extracted from the image file according to the template information; and integration information related to a medical record of a user is generated according to the text information.

The embodiment of the disclosure further provides an image content extraction device, which includes an image input interface and a processor. The image input interface is configured to obtain an image file. The processor is coupled to the image input interface. The processor is configured to analyze the image file to obtain distribution information of at least one grid in an image frame corresponding to the image file. The processor is further configured to determine template information according to the distribution information of the at least one grid. The processor is further configured to extract text information from the image file according to the template information. The processor is further configured to generate integration information related to a medical record of a user according to the text information.

Based on the above, after an image file is obtained, the image file may be analyzed to obtain distribution information of at least one grid in an image frame corresponding to the image file. Next, template information may be determined according to the distribution information of the at least one grid, and text information may be extracted from the image file according to the template information. Thereafter, integration information related to a medical record of a user may be automatically generated based on the text information. Accordingly, the efficiency of the use of printed certificates of diagnosis may be effectively improved.

### BRIEF DESCRIPTION OF THE DRAWINGS

FIG. 1 is a functional block diagram of an image content extraction device according to an embodiment of the disclosure.

FIG. 2 is a schematic diagram of an image frame corresponding to an image file according to an embodiment of the disclosure.

FIG. 3 is a schematic diagram of a pre-processed image frame according to an embodiment of the disclosure.

FIG. 4 is a schematic diagram of first distribution information and second distribution information according to an embodiment of the disclosure.

FIG. 5 is a schematic diagram of integration information according to an embodiment of the disclosure.

FIG. 6 is a schematic diagram of the appearance of an image content extraction device according to an embodiment of the disclosure.

FIG. 7 is a flow chart of an image content extraction method according to an embodiment of the disclosure.

### DETAILED DESCRIPTION OF DISCLOSED EMBODIMENTS

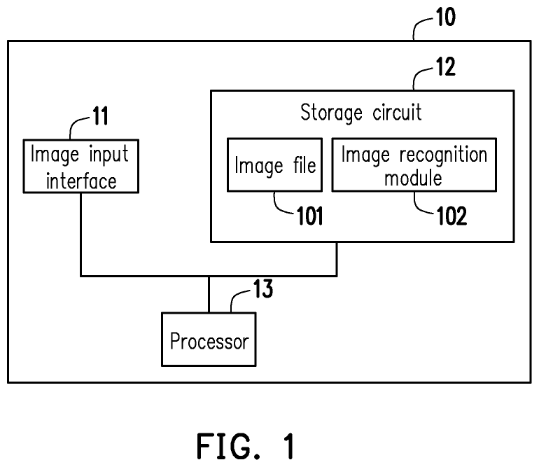

FIG. 1 is a functional block diagram of an image content extraction device according to an embodiment of the disclosure. Referring to FIG. 1, an image content extraction device 10 includes an image input interface 11, a storage circuit 12, and a processor 13. The image input interface 11 is configured to obtain an image file 101. The image file 101 may be generated by scanning a certificate of diagnosis. For example, the certificate of diagnosis may include a printed certificate of diagnosis issued by the hospital after a user (or a patient) receives medical treatment in a medical institution. This certificate of diagnosis may provide information related to the medical record of the user.

In an embodiment, the image input interface 11 may include an optical scanning device. This optical scanning device may scan a printed certificate of diagnosis through optical scanning to generate the image file 101. The file content of this image file 101 may reflect the content recorded on the certificate of diagnosis. Alternatively, in an embodiment, the image input interface 11 may include a file transfer interface. The file transfer interface may receive the image file 101 from the Internet or from any storage medium (such as a flash drive).

The storage circuit 12 is configured to store data (including the image file 101). For example, the storage circuit 12 may include a volatile storage circuit and a nonvolatile storage circuit. The volatile storage circuit is configured to store data in a volatile manner. For example, the volatile storage circuit may include a random access memory (RAM) or a similar volatile storage medium. The nonvolatile storage circuit is configured to store data in a nonvolatile manner. For example, the nonvolatile storage circuit may include a read only memory (ROM), a solid state disk (SSD), and/or a hard disk drive (HDD), or a similar nonvolatile storage medium.

The processor 13 is coupled to the image input interface 11 and the storage circuit 12. The processor 13 is configured to control the entirety or part of the operation of the image content extraction device 10. For example, the processor 13 may include a central processing unit (CPU), or other programmable general-purpose or special-purpose devices such as a micro processor, a digital signal processors (DSP), a programmable controller, an application specific integrated circuit (ASIC), a programmable logic device (PLD), or other similar devices, or a combination of the devices described above.

In an embodiment, the storage circuit 12 further stores an image recognition module 102. The image recognition module 102 may execute an image recognition operation such as a machine vision operation. For example, the processor 13 may run the image recognition module 102 to automatically recognize a specific object presented in the image frame corresponding to the image file 101. In addition, the image recognition module 102 may also be trained to improve recognition accuracy.

In an embodiment, the image recognition module 102 may also be implemented as a hardware circuit. For example, the image recognition module 102 may be implemented as an independent image processing chip (such as a GPU). Alternatively, the image recognition module 102 may also be disposed inside the processor 13. In an embodiment, the image content extraction device 10 may further include various signal output/output devices of such as a communication interface, a mouse, a keyboard, a screen, a touch screen, a speaker, and/or a microphone, and the disclosure is not limited thereto.

In an embodiment, the processor 13 may obtain the image file 101. For example, the processor 13 may read the image file 101 from the storage circuit 12. Next, the processor 13 may analyze the image file 101 through the image recognition module 102 and automatically generate integration information related to the medical records of a user based on the analysis result.

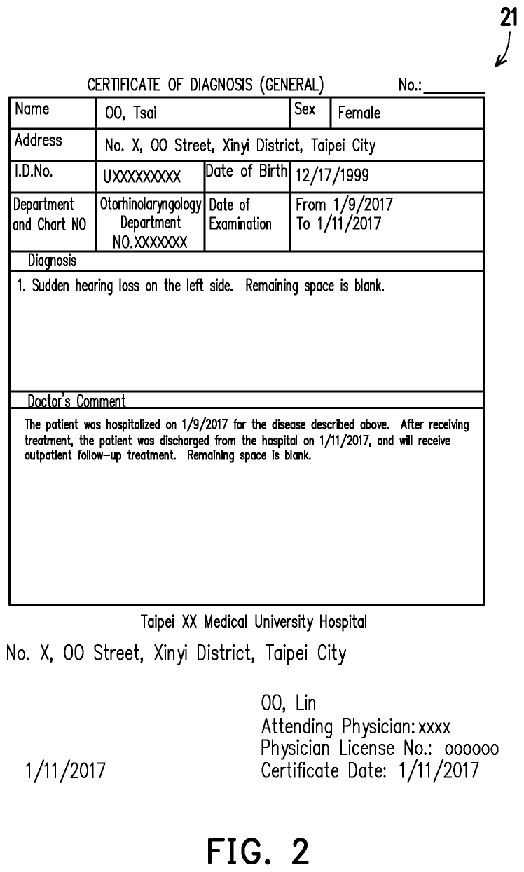

FIG. 2 is a schematic diagram of an image frame corresponding to an image file according to an embodiment of the disclosure. Referring to FIG. 2, taking an image frame 21 as an example of the image frame corresponding to the image file 101, the image frame 21 presents information related to the medical records of a user (that is, a patient). For example, the information presented in the image frame 21 may include the personal information of the user (such as name, sex, residential address, ID number, and date of birth), information of the medical records (such as department, chart number, and date of examination), and diagnosis information (such as illness, doctor's comment, attending physician), etc., as shown in FIG. 2.

It is to be noted that what is shown in FIG. 2 is an example of an information recording format (including the form format) of the certificate of diagnosis provided by the Taipei XX Medical University Hospital. However, the information recording format of certificates of diagnosis used by different medical institutions may be different (for example, the total number of fields in the form, the contents recorded in the fields, and/or the arrangement of the fields may be different).

In an embodiment, if the information recording format of the certificate of diagnosis corresponding to the image file 101 obtained at the moment cannot be confirmed, even if the processor 13 (or the image recognition module 102) supports an image recognition function, the information automatically recognized by the processor 13 (or the image recognition module 102) from the image file 101 (or image frame 21) may still not be classified and filed correctly. For example, before obtaining the configuration logic and/or relevance of each field in the image frame 21, even if information such as “Name” and “OO, Tsai” are obtained from the image frame 21 through an image recognition technology, the processor 13 may still not be able to combine the information into correct and understandable integration information (such as “Name: OO, Tsai”) or similar information).

In an embodiment, the processor 13 may analyze the image file 101 to obtain distribution information of at least one grid in the image frame corresponding to the image file 101. For example, the processor 13 may analyze the image file 101 through the image recognition module 102 to recognize at least one grid in the image frame 21 corresponding to the image file 101. Next, the processor 13 may generate the distribution information of the grid according to the result of the image recognition module 102 recognizing the grid. In the following embodiments, the image frame 21 is used as the image frame corresponding to the image file 101, but the disclosure is not limited thereto.

In an embodiment, before analyzing the distribution of the grid in the image frame 21, the processor 13 may execute pre-processing on the image frame 21 to attempt to filter out an image content that does not belong to the grid in the image frame 21. For example, during pre-processing, the processor 13 may recognize straight line(s) of at least one direction in the image frame 21 through the image recognition module 102. Next, the processor 13 may retain the recognized line(s), and filter out the image content other than the line(s).

In an embodiment, during pre-processing on the image frame 21, the processor 13 may first filter out a color content in the image frame 21. For example, the color content may include a stamp of the hospital and/or a personal stamp of the attending physician on the original printed certificate of diagnosis. Filtering out the color content may help improve the accuracy of the subsequent grid analysis. In an embodiment, after filtering out the color content in the image frame 21, the image frame 21 may only contain a black image content (the grid and the text) and a white image content (the background). Next, the processor 13 may filter out the image content that does not belong to the grid in the image frame 21. Next, the processor 13 may analyze the pre-processed image frame to obtain the distribution information of the at least one grid.

It is to be noted that in an embodiment, the processor 13 may directly detect the grid in the original image frame 21 without executing pre-processing on the image frame 21 to conserve computing resources of the system. However, the accuracy of grid detection may thus decrease.

In an embodiment, the processor 13 may obtain the distribution information (or first distribution information) of at least one grid (or a first grid) parallel to a direction (or a first direction) in the image frame 21. In addition, the processor 13 may obtain the distribution information (or second distribution information) of at least one grid (or a second grid) parallel to another direction (or a second direction) in the image frame 21. The first direction may be perpendicular to the second direction. For example, in an embodiment, the first direction may be a horizontal direction, and the second direction may be a vertical direction. Alternatively, in an embodiment, the first direction may be a vertical direction, and the second direction may be a horizontal direction, as long as the two directions are perpendicular to each other.

In an embodiment, the processor 13 may scan the image frame 21 along the first direction from a coordination position (or a first coordination position) in the image frame 21 and record the total number of at least one characteristic point (or at least one first characteristic point) detected. The processor 13 may generate the first distribution information according to the total number of the first characteristic point. In addition, the processor 13 may scan the image frame 21 along the second direction from a coordination position (or a second coordination position) in the image frame 21, and record the total number of at least one characteristic point (or at least one second characteristic point) detected. The processor 13 may generate the second distribution information according to the total number of the second characteristic point.

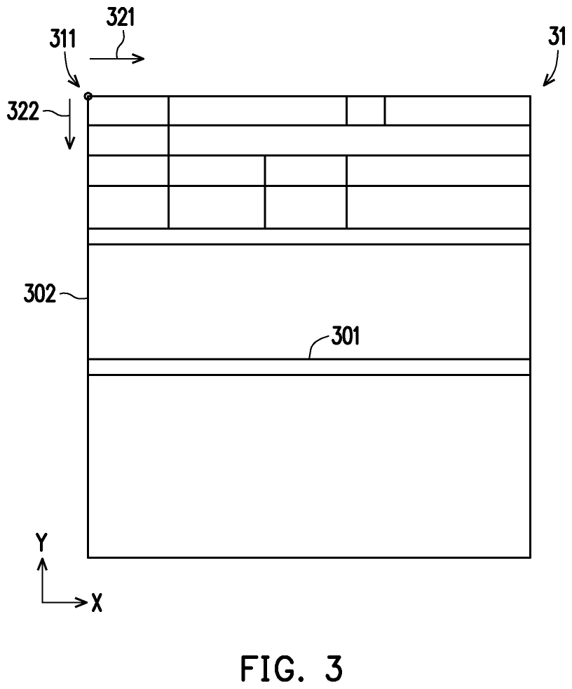

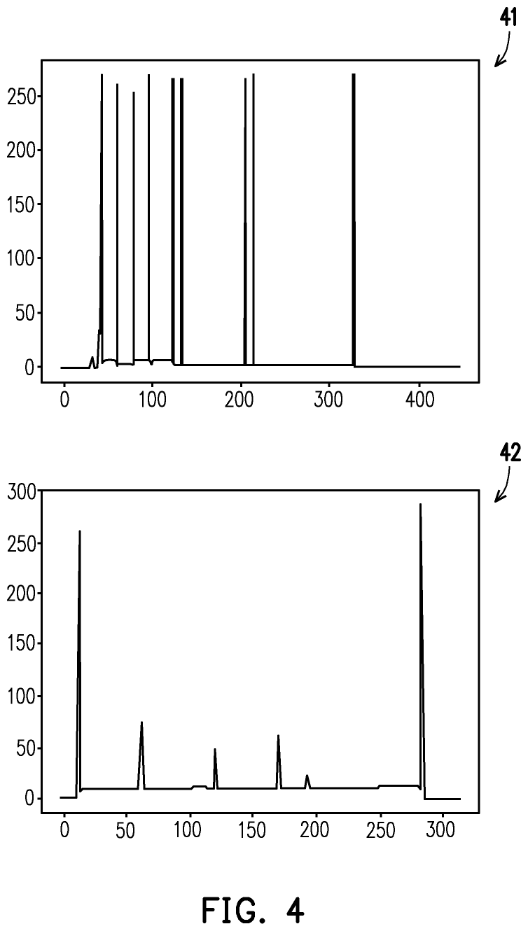

FIG. 3 is a schematic diagram of a pre-processed image frame according to an embodiment of the disclosure. FIG. 4 is a schematic diagram of first distribution information and second distribution information according to an embodiment of the disclosure. Referring to FIG. 3, following the embodiment of FIG. 2, the processor 13 may execute pre-processing on the original image frame 21 to generate a new image frame 31. Compared with the image frame 21, most of the text and/or graphics (except for the straight lines) in the image frame 31 are viewed as noise that may affect the recognition result and are thus filtered out. Only multiple grids 301 and multiple grids 302 are retained. For example, the grids 301 refer to grids parallel to the X-axis direction (that is, the first direction or the horizontal direction), and the grids 302 refer to grids parallel to the Y-axis direction (that is, the second direction or the vertical direction).

In an embodiment, the processor 13 may determine a coordination position in the recognized grids 301 and 302, and determine the coordination position as the first coordination position. Taking a coordination position 311 as an example of the first coordination position, the coordination position 311 may be located at the leftmost end of a form region formed by the grids 301 and the grids 302. The processor 13 may scan the image frame 31 along a direction 321, starting from the coordination position 311, (that is, the first direction or the X-axis direction), and record the total number of the at least one characteristic point (that is, the first characteristic point) detected. For example, the first characteristic point is a black pixel point detected in the image frame 31 along the X-axis direction. The processor 13 may move downward from the coordination position 311 (that is, along the Y-axis direction or a direction 322) and sequentially record the total number of the first characteristic point detected by scanning to the right. The processor 13 may generate distribution information 41 in FIG. 4 according to the total number of the first characteristic point detected.

Referring to FIG. 4, the horizontal axis of the distribution information 41 corresponds to a Y-axis coordinate of the image frame 31, and the vertical axis of the distribution information 41 corresponds to the total number of the first characteristic point measured by scanning to the right from a specific Y-axis coordinate in the image frame 31. In other words, the distribution information 41 may reflect the distribution state of the grids 301 on the image frame 31 through a statistical characteristic.

On the other hand, taking the same coordination position 311 as an example of the second coordination position, the processor 13 may scan the image frame 31 along the direction 322 (that is, the second direction or the Y-axis direction) starting from the coordination position 311, and record the total number of at least one characteristic point (that is, the second characteristic point) detected. For example, the second characteristic point is a black pixel point detected in the image frame 31 along the Y-axis direction. The processor 13 may move from the coordination position 311 towards the right (that is, along the X-axis direction or the direction 321) and sequentially record the total number of the second characteristic point detected by scanning downwards. The processor 13 may generate distribution information 42 in FIG. 4 according to the total number of the second characteristic point detected.

Referring to FIG. 4, the horizontal axis of the distribution information 42 corresponds to an X-axis coordinate of image frame 31, and the vertical axis of the distribution information 42 corresponds to the total number of the second characteristic point measured by scanning downwards from a specific X-axis coordinate on image frame 31. In other words, the distribution information 42 may reflect the distribution state of the grids 302 on the image frame 31 through a statistical characteristic.

In an embodiment, the distribution information 41 and 42 may be separately or collectively referred to as statistical distribution information of the grids. The statistical distribution information of the grids may serve as a statistical characteristic to reflect the distribution state of the grids on the image frame 21 or the image frame 31.

Referring back to FIG. 3, in an embodiment, during the process of scanning the entire image frame 31, the processor 13 may further obtain actual distribution information of the grids. The actual distribution information of the grids may reflect the (actual) distribution state of multiple fields divided by the grids 301 and the grids 302 on the image frame 31. For example, compared with the statistical distribution information of the grids (such as the distribution information 41 and the distribution information 42), the actual distribution information of the grids may present information related to the actual distribution state of the fields, such as the actual positions of the fields divided by the grids 301 and the grids 302 in the image frame 31, a pixel range actually covered by the fields, and/or the relative positions between the fields.

In an embodiment, during the process of scanning the image frame 21 or the image frame 31, the processor 13 may continuously record the coordination position(s) of the first characteristic point and/or the second characteristic point. The processor 13 may depict the fields and the actual distribution state thereof according to the coordination position(s) of the first characteristic point and/or the second characteristic point. The processor 13 may obtain the actual distribution information of the grids based on the information described above.

In an embodiment, after obtaining the distribution information of the grids, the processor 13 may determine template information according to the distribution information of the grids. The template information may be configured to extract required text information from the image frame 21. For example, the processor 13 may compare the statistical distribution information of the grids with grid distribution information of at least one candidate template in the storage circuit 12. The processor 13 may determine the template information according to the comparison result.

Taking FIG. 4 as an example, the processor 13 may compare the distribution information 41 and/or the distribution information 42 with grid distribution information of at least one candidate template in a database. If the comparison result shows that the similarity of the distribution of the grids in the image frame 21 (or the image frame 31) and the distribution of the grids of a candidate template is higher than a threshold (for example, the similarity of the distribution of the grids is higher than 90%), the processor 13 may determine the candidate template as a target template and read the template information of the target template from the storage circuit 12. For example, the processor 13 may read the template information of the certificate of diagnosis provided by the Taipei XX Medical University Hospital from the storage circuit 12 according to the comparison result. The template information may reflect the information recording format of the certificate of diagnosis assumedly provided by the Taipei XX Medical University Hospital.

In an embodiment, if the template information automatically picked by the processor 13 is incorrect, the user may execute a user operation to adjust (for example, replace) the template information automatically picked by the processor 13. For example, in an embodiment, if the user discovers that the template information automatically determined by the processor 13 after the processor 13 analyzes the image frame 21 is incorrect (for example, the template information a certificate of diagnosis of another hospital is applied), the user may execute the user operation through an input/output interface of the image content extraction device 10 (such as a mouse, a touchpad, or a touch screen), so as to select the correct template information from the database. The processor 13 may apply the correct template information according to the user operation described above to avoid errors in a subsequent text extraction.

In an embodiment, the processor 13 may record the information of a selected incorrect template information. The processor 13 may modify certain algorithm parameters (for example, the weight information of an artificial intelligence algorithm) used in a subsequent template information selection according to the information, so as to attempt to increase the accuracy of the subsequent template information selection. For example, in an embodiment, the processor 13 may increase or decrease the probability of selecting template information according to the modified algorithm parameters (for example, the weight information).

In an embodiment, after determining the template information, the processor 13 may extract text information from the image frame 21 according to the template information. For example, in an embodiment, the processor 13 may extract specific text information (or first text information) from at least one preset field among the divided fields according to the actual distribution information of the grids and the template information.

Taking FIG. 2 as an example, according to the template information corresponding to the certificate of diagnosis of the Taipei XX Medical University Hospital and the actual distribution information of the grids, the processor 13 may extract information of the name of a user and information of the sex of the user from the second column of the first layer and the fourth column of the first layer in the image frame 21, respectively. Later, during the process of generating integration data, the text information extracted from the second column of the first layer in the image frame 21 may be matched to the item content of “name” in the integrated data, the text information extracted from the fourth column of the first layer in the image frame 21 may be matched to the item content of “sex” in the integrated data, and so forth.

Alternatively, in an embodiment, the processor 13 may use keyword searching to match the template information to extract specific text information (or second text information) from the image frame 21. Taking FIG. 2 as an example, again, according to the template information corresponding to the certificate of diagnosis of the Taipei XX Medical University Hospital, the processor 13 may search for the keyword “attending physician” in the image frame 21 and extract the information of the physician from what follows the keyword, and so forth. Later, during the process of generating the integration data, the text information (such as OO, Lin) extracted from what follows the keyword may be matched to the item content of “diagnosing physician” in the integration data.

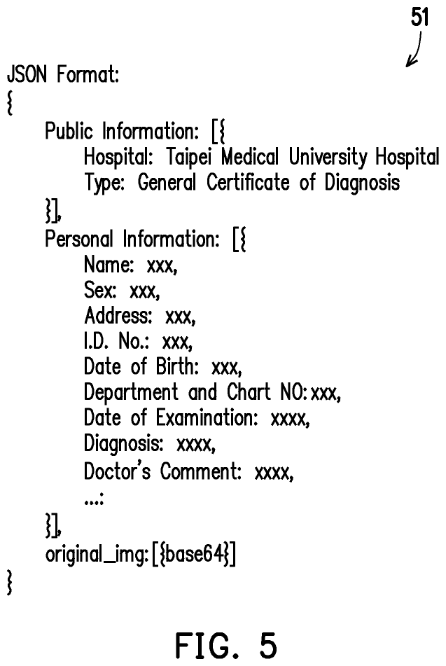

FIG. 5 is a schematic diagram of integration information according to an embodiment of the disclosure. Referring to FIG. 5, in an embodiment, after extracting the required text information, the processor 13 may generate integration information 51 related to the medical record of the user according to the extracted text information. For example, the integration information 51 may classify and record the item content of each field presented in the form in the original image frame 21 in a computer-readable text format. The item content may include, for example, the personal information of the user (such as the name, sex, residential address, ID number, date of birth), information of the medical records (such as department, chart number, and date of examination), and diagnosis information (such as illness, doctor's comment, attending physician), etc.

In an embodiment, if the text information extracted by the processor 13 is incorrect, the user may execute a user operation to modify the text information extracted by the processor 13. For example, in an embodiment, if the user discovers that the text information extracted by processor 13 is incorrect (for example, incorrectly recognizing a specific text and/or mismatching specific information) (for example, matching the age item to the name of the user)), the user may execute the user operation through the input/output interface of the image content extraction device 10 (such as a mouse, a touchpad, or a touch screen) so as to correct the error. The processor 13 may generate a modification record of a user according to the user operation and send the modification record back to the database. Later, according to the modification record, back-end management personnel (such as maintenance personnel or insurance company personnel) may know that the user has modified a recognized specific text information and receive a corresponding modification result.

In an embodiment, the processor 13 may modify certain algorithm parameters (for example, the weight information of an artificial intelligence algorithm) that are configured to subsequently recognize the text information according to the modification record of the user of the text information, so as to attempt to improve the correctness of the subsequent text information recognition.

It is to be noted that in other embodiments, the template information may further contain other useful information (for example, descriptions related to blank characters and/or special symbols that may exist in the image frame 21). Accordingly, the processor 13 may automatically and completely extract the qualified text information from the image frame 21 according to the information described above, and generate the integration information 51 related to the medical record of the user according to the text information.

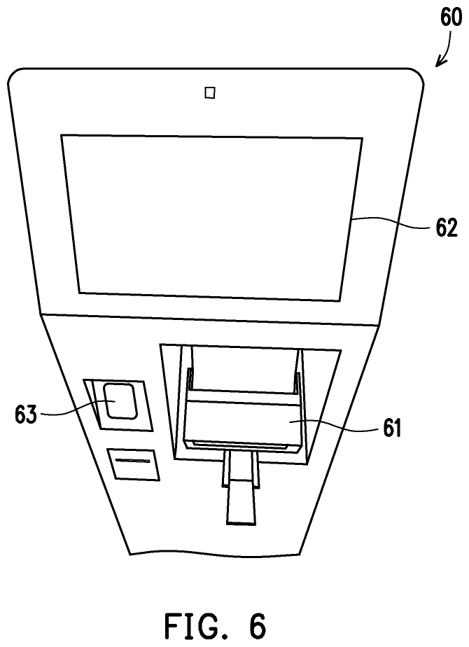

FIG. 6 is a schematic diagram of the appearance of an image content extraction device according to an embodiment of the disclosure. Referring to FIG. 6, an image content extraction device 60 may be the same or similar to the image content extraction device 10. In an embodiment, the image content extraction device 60 may include an image input interface 61 and a display interface 62. The image input interface 61 may be configured to receive printed certificates of diagnosis. For example, the image input interface 61 may include a paper inserting entrance. The user may insert the printed certificate of diagnosis into the image input interface 61 and enable the image input interface 61 to generate an image file corresponding to the certificate of diagnosis.

The display interface 62 may be a touch screen and configured to display the template information automatically determined by the processor of the image content extraction device 60. In an embodiment, the user may check whether the template information is correct from the display interface 62. If the template information is incorrect, the user may select the correct template information to replace the incorrect template information by operating the display interface 62 by touch. In addition, in an embodiment, the user may view and/or modify the text information and/or the integration information automatically extracted by the processor of the image content extraction device 60 through the display interface 62.

In an embodiment, the image content extraction device 60 may further include an authentication interface 63. The authentication interface 63 may be configured to verify the identity of a current operator. For example, the user may place their ID card close to the authentication interface 63 to achieve identity verification by means of wireless sensing or QR code scanning.

In an embodiment, the image content extraction device 60 may further include a communication interface (not shown). The communication interface may be configured to send the generated image file and/or integration information to a remote device, such as a mobile phone of a specific user or a server of an insurance company, etc., for a subsequent inquiry.

It is to be noted that the appearance of the image content extraction device 60 in FIG. 6 only serves as an example. In an embodiment, the appearance and/or interface configuration of the image content extraction device 60 may be adjusted according to actual needs, and the disclosure is not limited thereto.

In an embodiment, the image content extraction device 60 in FIG. 6 (or the image content extraction device 10 in FIG. 1) may be implemented as various computer devices with an image processing function, such as a notebook computer, a desktop computer, an industrial computer, a server, a smart phone, or a tablet computer, and the disclosure does not limit the types of the image content extraction device 10 and the image content extraction device 60.

In addition, an embodiment of the disclosure proposes a nonvolatile computer-readable recording medium. This nonvolatile computer-readable recording medium stores a code. The processor in the computer (such as the processor 13 in FIG. 1) may execute (or run) the code to execute the functions and operations described above.

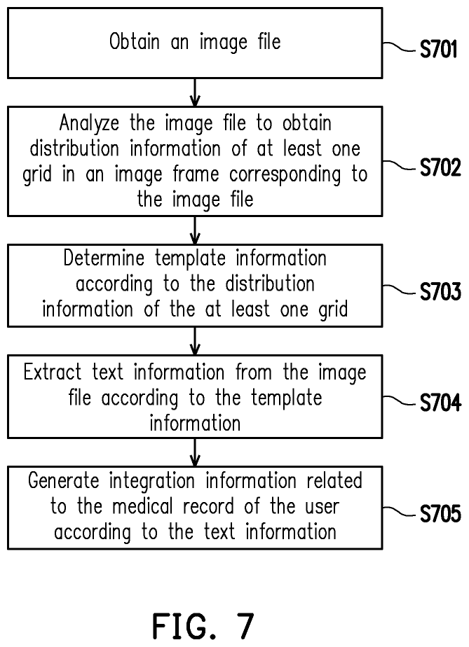

FIG. 7 is a flow chart of an image content extraction method according to an embodiment of the disclosure. Referring to FIG. 7, in step S701, an image file is obtained. In step S702, the image file is analyzed to obtain distribution information of at least one grid in an image frame corresponding to the image file. In step S703, template information is determined according to the distribution information of the at least one grid. In step S704, text information is extracted from the image file (or the image frame) according to the template information. In step S705, integration information related to the medical record of the user is generated according to the text information.

However, each step in FIG. 7 has been described in detail as above, and will not be repeated herein. It is to be noted that each step in FIG. 7 may be implemented as multiple codes (such as a software module) or a circuit (such as a circuit module), and the disclosure is not limited thereto. In addition, the method in FIG. 7 may be used along with the embodiments described above or used alone, and the disclosure is not limited thereto.

In summary, after the image file is obtained, the image file may be analyzed to obtain the distribution information of the at least one grid in the image frame corresponding to the image file. Next, the template information may be determined according to the distribution information of the at least one grid, and the text information may be extracted from the screen of the image file according to the template information. Thereafter, the integration information related to the medical record of the user may be automatically generated according to the text information. Accordingly, efficiency of using the printed certificate of diagnosis may be effectively improved.

Although the disclosure has been disclosed in the above embodiments, the embodiments not intended to limit the disclosure. Those skilled in the art can make some changes and modifications without departing from the spirit and scope of the disclosure. Therefore, the scope of the disclosure is defined by the claims that follow.

What is claimed is:

1. An image content extraction method, comprising:
obtaining an image file;
analyzing the image file to obtain distribution information of at least one grid in an image frame corresponding to the image file;
determining template information according to the distribution information of the at least one grid;
extracting text information from the image frame according to the template information; and
generating integration information related to a medical record of a user according to the text information.

2. The image content extraction method according to claim 1, wherein the step of analyzing the image file to obtain the distribution information of the at least one grid in the image frame corresponding to the image file comprises:
filtering out a color content in the image frame.

3. The image content extraction method according to claim 1, wherein the step of analyzing the image file to obtain the distribution information of the at least one grid in the image frame corresponding to the image file comprises:
filtering out an image content that does not belong to the at least one grid in the image frame.

4. The image content extraction method according to claim 1, wherein the step of analyzing the image file to obtain the distribution information of the at least one grid in the image frame corresponding to the image file comprises:
obtaining first distribution information of at least one first grid parallel to a first direction in the at least one grid; and
obtaining second distribution information of at least one second grid parallel to a second direction in the at least one grid, wherein the first direction is perpendicular to the second direction.

5. The image content extraction method according to claim 4, wherein the step of obtaining the first distribution information of the at least one first grid parallel to the first direction in the at least one grid comprises:
scanning the image frame along the first direction from a first coordination position in the image frame and recording the total number of at least one first characteristic point detected; and
generating the first distribution information according to the total number of the at least one first characteristic point,
wherein the operation of obtaining the second distribution information of the at least one second grid parallel to the second direction in the at least one grid comprises:
scanning the image frame along the second direction from a second coordination position in the image frame and recording the total number of at least one second characteristic point detected; and
generating the second distribution information according to the total number of the at least one second characteristic point.

6. The image content extraction method according to claim 1, wherein the distribution information of the at least one grid comprises statistical distribution information of the at least one grid, and the statistical distribution information of the at least one grid reflects a distribution state of the at least one grid on the image frame through a statistical characteristic.

7. The image content extraction method according to claim 6, wherein the step of determining the template information according to the distribution information of the at least one grid comprises:
comparing the statistical distribution information of the at least one grid with grid distribution information of at least one candidate template; and
determining the template information according to a comparison result.

8. The image content extraction method according to claim 1, wherein the distribution information of the at least one grid comprises actual distribution information of the at least one grid, and the actual distribution information of the at least one grid reflects a distribution state of a plurality of fields divided by the at least one grid on the image frame.

9. The image content extraction method according to claim 8, wherein the step of extracting the text information from the image frame according to the template information comprises:
extracting first text information from at least one preset field in the plurality of fields according to the actual distribution information of the at least one grid and the template information.

10. The image content extraction method according to claim 1, wherein the step of extracting the text information from the image frame according to the template information comprises:
using keyword searching to match the template information so as to extract second text information from the image frame.

11. An image content extraction device, comprising:
an image input interface, configured to obtain an image file;
a processor, coupled to the image input interface,
wherein the processor is configured to analyze the image file to obtain distribution information of at least one grid in an image frame corresponding to the image file,
the processor is further configured to determine template information according to the distribution information of the at least one grid,
the processor is further configured to extract text information from the image frame according to the template information, and
the processor is further configured to generate integration information related to a medical record of a user according to the text information.

12. The image content extraction device according to claim 11, wherein the operation of analyzing the image file to obtain the distribution information of the at least one grid in the image frame corresponding to the image file comprises:
filtering out a color content in the image frame.

13. The image content extraction device according to claim 11, wherein the operation of analyzing the image file to obtain the distribution information of the at least one grid in the image frame corresponding to the image file comprises:
filtering out an image content that does not belong to the at least one grid in the image frame.

14. The image content extraction device according to claim 11, wherein the operation of analyzing the image file to obtain the distribution information of the at least one grid in the image frame corresponding to the image file comprises:
obtaining first distribution information of at least one first grid parallel to a first direction in the at least one grid; and
obtaining second distribution information of at least one second grid parallel to a second direction in the at least one grid, wherein the first direction is perpendicular to the second direction.

15. The image content extraction device according to claim 14, wherein the operation of obtaining the first distribution information of the at least one first grid parallel to the first direction in the at least one grid comprises:
scanning the image frame along the first direction from a first coordination position in the image frame and recording the total number of at least one first characteristic point detected; and
generating the first distribution information according to the total number of the at least one first characteristic point,
wherein the operation of obtaining the second distribution information of the at least one second grid parallel to the second direction in the at least one grid comprises:
scanning the image frame along the second direction from a second coordination position in the image frame and recording the total number of at least one second characteristic point detected; and
generating the second distribution information according to the total number of the at least one second characteristic point.

16. The image content extraction device according to claim 11, wherein the distribution information of the at least one grid comprises statistical distribution information of the at least one grid, and the statistical distribution information of the at least one grid reflects a distribution state of the at least one grid on the image frame through a statistical characteristic.

17. The image content extraction device according to claim 16, wherein the operation of determining the template information according to the distribution information of the at least one grid comprises:
comparing the statistical distribution information of the at least one grid with grid distribution information of at least one candidate template; and
determining the template information according to a comparison result.

18. The image content extraction device according to claim 11, wherein the distribution information of the at least one grid comprises actual distribution information of the at least one grid, and the actual distribution information of the at least one grid reflects a distribution state of a plurality of fields divided by the at least one grid on the image frame.

19. The image content extraction device according to claim 18, wherein the operation of extracting the text information from the image frame according to the template information comprises:
extracting first text information from at least one preset field in the plurality of fields according to the actual distribution information of the at least one grid and the template information.

20. The image content extraction device according to claim 11, wherein the operation of extracting the text information from the image frame according to the template information comprises:
using keyword searching to match the template information so as to extract second text information from the image frame.
